(19) United States
(12) Patent Application Publication
(10) Pub. No.: US 2026/0345149 A1
(43) Pub. Date: Nov. 5, 2026

(54) IMPLANTABLE MEDICAL DEVICE DEPLOYMENT DEVICES, SYSTEMS, AND METHODS

(71) Applicant: Boston Scientific Scimed, Inc., Maple Grove, MN (US)

(72) Inventors: Timothy Noah Foster-Frau, Austin, TX (US); Jason N. Mehta, Austin, TX (US); Alexander R. Cohen, Austin, TX (US); John A. Mims, Austin, TX (US)

(73) Assignee: Boston Scientific Scimed, Inc., Maple Grove, MN (US)

(21) Appl. No.: 19/663,491

(22) Filed: Apr. 30, 2026

Related U.S. Application Data
(60) Provisional application No. 63/798,072, filed on May 1, 2025.

(51) Int. Cl. A61B 17/04; A61B 17/00

(52) U.S. Cl. CPC A61B 17/0401; A61B 17/00234; A61B 2017/00296; A61B 2017/00367; A61B 2017/00424; A61B 2017/00477; A61B 2017/0409

(57) ABSTRACT

A deployment device which is configured to be operated by a single medical professional to advance, engage, deploy, and release an implantable medical device at an implant site within a patient’s body. In some aspects, the deployment device is configured for single-handed use to advance an implantable medical device into the patient’s body as well as to deploy and separate the implantable medical device from the deployment device within the patient’s body.

### CROSS-REFERENCE TO RELATED APPLICATIONS

This application claims the benefit of priority under 35 U.S.C. § 119 to U.S. Provisional Application No. 63/798,072, filed on May 1, 2025, the entire disclosure of which is hereby incorporated by reference herein for all purposes.

### FIELD

The present disclosure relates generally to the field of medical devices, systems, and methods for deploying an implantable medical device. More particularly, the present disclosure relates to medical devices, systems, and methods which facilitate deployment of an implantable medical device with respect to a deployment site, such as by a single user and/or using only one hand.

### BACKGROUND

Endoscopy is a complex and often ergonomically challenging enterprise. For many operations, the medical professional who is primarily responsible for performing the procedure (e.g., a physician) will be assisted by another medical professional who may control one or more accessories to the endoscope (e.g., fluid supply, vacuum pressure, a lighting or visualization device, a device to be extended through a working channel, etc.). For instance, the other medical professional may assist the physician with using an endoscopic device to be inserted through the working channel of the endoscope, such as assistant-driven snares, cinches, graspers, and various other devices. While having a medical assistant drive a device is commonplace, the physician loses tactile feedback and/or fine control of the device, and must focus on communication with the assistant as well as on the procedure itself. When deploying an implantable medical device at an implant site, a physician will drive the endoscope and put forward pressure on a delivery device to engage the implantable medical device with the implant site. The physician will then request a medical assistant to manipulate the implantable medical device itself in order to be implanted with respect to the implant site. Various delivery devices and systems for deploying implantable medical devices must be operated to achieve the proper implantation of the implantable medical device. For instance, in the case of a deployable helical anchor, once the physician achieves initial contact of the anchor with the implant site (by advancing the endoscope and the device to the implant site), the helical anchor must be rotated to be further advanced into tissue. The deployment device and/or system must then be further manipulated (e.g., by manipulating user-engagement features or controls on a control handle) to deploy the helical anchor by separating the helical anchor from the deployment device and/or system to be left in place, implanted with respect to the implant site. Typically, the medical assistant is the one who operates the deployment device and/or system, and stands a comfortable distance away from the physician performing the procedure. By depending on the medical assistant, the physician loses tactile feedback from the deployment device and/or system, which may lead to a decrease in confidence and, consequently, speed.

There remains a need for providing better feedback and/or control to a medical professional when using a medical instrument to perform a procedure. Solutions to these and other challenges in the art would be welcome, and it is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In some aspects, a deployment device for deploying an implantable medical device at an implant site within a patient’s body includes, in accordance with various principles of the present disclosure, a driver flexible elongate element with a distal end including a driver configured to be operably coupled with an implantable medical device to deliver the implantable medical device to the implant site; a separator element adjacent the driver and movable with respect to the driver to act upon the implantable medical device to separate the implantable medical device from the driver for deployment at the implant site; and an actuator flexible elongate element operably coupled with respect to the driver flexible elongate element to move axially and rotationally with the driver flexible elongate element, and axially movable with the separator element to advance the separator element to the implant site. In some aspects, at least a portion of the actuator flexible elongate element is axially movable with respect to the separator element to cause relative movement between the driver and the separator element to cause separation of the implantable medical device from the driver.

In some aspects, the actuator flexible elongate element is operably coupled with respect to the separator element to move the separator element axially, and additionally is axially movable with respect to the separator element.

In some aspects, the actuator flexible elongate element is elongatable to move axially with respect to the separator element.

In some aspects, the actuator flexible elongate element is stretchable to move axially with respect to the separator element.

In some aspects, the deployment device further includes a control handle operably coupled with the actuator flexible elongate element. In some aspects, the driver flexible elongate element is operably coupled with the actuator flexible elongate element to be operably coupled with the control handle such that movement of the control handle moves the driver flexible elongate element. In some aspects, a proximal portion of the actuator flexible elongate element adjacent and distal to the control handle is manually accessible for manual movement to cause movement of the driver flexible elongate element. In some aspects, the control handle and the proximal portion of the actuator flexible elongate element are accessible in a single hand of a user.

In some aspects, the actuator flexible elongate element is rotatably coupled with respect to the separator element to rotate with respect to the separator element. In some aspects, the actuator flexible elongate element is rotatably coupled with respect to the separator element via a coupler, and the at least a portion of the actuator flexible elongate element is axially movable with respect to the coupler.

In some aspects, a deployment system for deploying an implantable medical device at an implant site within a patient’s body includes, in accordance with various principles of the present disclosure, an actuator flexible elongate element having a proximal end and a distal end; a driver operably coupled with respect to the distal end of the actuator flexible elongate element to move axially and/or rotationally with the actuator flexible elongate element; and a separator element adjacent the driver. In some aspects, the proximal end of the actuator flexible elongate element is manually manipulable in a single hand of a user in more than one direction to advance and deploy an implantable medical device from the driver, as well as to cause the implantable medical device to separate from the driver to be left at the implantable medical device implanted with respect to the implant site within the patient’s body.

In some aspects, the deployment system further includes a control handle operably coupled with the proximal end of the actuator flexible elongate element and sized to fit within a user’s hand and allow the user’s fingers to contact a proximal portion of the actuator flexible elongate element adjacent and distal to the control handle. In some aspects, the control handle and the portion of the actuator flexible elongate element adjacent and distal to the control handle are accessible in a single hand of a user.

In some aspects, the deployment system further includes a brake accessory operably coupled with the actuator flexible elongate element to fix a portion of the actuator flexible elongate element distal to the control handle against axial movement with respect to the separator element. In some aspects, the deployment system further includes a medical delivery device having a port through which the driver, the separator element, and the distal end of the actuator flexible elongate element are deliverable into a patient’s body. In some aspects, the brake accessory is mounted with respect to the port for operation by a user’s first hand operating the medical delivery device while the proximal end of the actuator flexible elongate element is graspable in the user’s second hand for operation of the actuator flexible elongate element.

In some aspects, a method of deploying an implantable medical device at an implant site within a patient’s body includes, in accordance with various principles of the present disclosure, distally advancing the implantable medical device, operably coupled to a distal end of a driver of a deployment device, into a patient; engaging the implantable medical device with the implant site; deploying the implantable medical device with respect to the implant site; and releasing the implantable medical device from the driver. In some aspects, advancing, engaging, deploying, and releasing are all performed by a single medical professional.

In some aspects, advancing, engaging, deploying, and releasing are all performed by a single hand of a single medical professional. In some aspects, the method further includes advancing the implantable medical device through a medical delivery device and into the patient; controlling the medical delivery device with a first hand of a single medical professional; and controlling the driver of the deployment device with a second hand of the single medical professional.

In some aspects, the method further includes axially advancing an actuator flexible elongate element with respect to a separator element positioned adjacent the driver, and axially moving the actuator flexible elongate element with respect to the separator element using the same hand. In some aspects, the method further includes advancing the implantable medical device into the patient’s body through a port of a medical delivery device, and holding a portion of an actuator flexible elongate element with the hand which holds the medical delivery device and a portion of the actuator flexible elongate element with the other hand, of the same medical professional, at least to release the implantable medical device from the driver.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a deployment system with a deployment device formed in accordance with various principles of the present disclosure, and usable with an additional medical delivery device, both devices being illustrated for use by a single medical professional such as to deploy one or more illustrated implantable medical devices.

FIG. 1A illustrates a cross-sectional view of a brake accessory usable in the deployment system illustrated in FIG. 1.

FIG. 2 illustrates an elevational view of a deployment device such as which may be used in the system illustrated in FIG. 1.

FIG. 2A illustrates a detail of FIG. 2.

FIG. 2B illustrates a detail of a distal end of the deployment device illustrated in FIG. 2 without an implantable medical device operably coupled therewith (such as in FIG. 2 and FIG. 2A).

FIG. 3A illustrates a cross-sectional view along line III-III of FIG. 2.

FIG. 3B illustrates a cross-sectional view similar to that of FIG. 3A, but in a second configuration.

FIG. 4 illustrates a cross-sectional view along line III-III of FIG. 2, but showing an alternate example of an embodiment of a coupler thereof.

FIG. 5 illustrates a perspective view of the example of an embodiment of a coupler illustrated in FIG. 4.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

There has been increasing interest in the medical field in minimally-invasive surgical procedures. One trend includes moving from laparoscopic and open surgical procedures to miniaturized transluminal / transcatheter procedures which do not require open surgery (cutting open the patient), but, instead, access a target site within the patient via a natural orifice (or, in some instances, a small incision not considered to constitute an open-surgery cut). For instance, minimally invasive procedures commonly utilize a medical scope, such as an endoscope, to access treatment sites within a patient’s body to allow a medical professional to perform ever more complex procedures noninvasively and under direct visualization. However, operation of the medical scope presents challenges in also operating various components of an additional instrument used to perform the procedure.

In accordance with various principles of the present disclosure, surgical and/or endoscopic devices and systems are configured to be driven / controlled by a single user, such as the physician primarily responsible for performing a medical / surgical procedure. A deployment device formed in accordance with various principles of the present disclosure is configured so that the medical professional operating a medical device (such as a medical scope) may also readily operate the deployment device without the need for assistance by another medical professional. Additionally or alternatively, the deployment device allows for single handed use, such as with a single hand of a medical professional who is operating the deployment device, such as the medical professional operating another device with his/her other hand. It will be appreciated that terms such as operate, control, drive, manipulate, maneuver, manipulate, move, navigate, etc., and other grammatical forms thereof, such as in relation to a medical device, may be usable interchangeably herein without intent to limit unless otherwise indicated. It will further be appreciated that terms such as instruments, tools, devices, accessories, etc., may be usable interchangeably herein without intent to limit unless otherwise indicated.

A deployment device formed in accordance with various principles of the present disclosure thus allows the physician who is performing a procedure with respect to a treatment site in a patient’s body to concentrate on and personally control the procedure. Moreover, the same individual who controls forward pressure of a device also controls further movements of the device, such as rotation, necessary to perform the procedure. The same medical professional can coordinate the “feel” of the tissue at the treatment site with visualization of the tissue and the further “feel” of the tissue upon deployment. Such coordination allows for finer control of linear adjustments and/or torque and thus lowers the risk of insufficient depth of placement of an implantable medical device (such as a tack). By introducing total physician control of a device and/or system, a medical / surgical procedure can be made safer and more efficient, with consequent reduction in procedure time, reduction in risks to the patient, and quicker recovery time. As may be appreciated, the present disclosure eliminates the various lags built into prior procedures, including the lag from the physician sensing when the next step in a procedure should be performed, the lag in effectively communicating to a medical assistant that the next step should be performed, and the lag in implementation of the next step by a medical assistant. The various demands of synchronicity and choreography between the physician who is performing the procedure and the medical assistant is no longer necessary for clinical success and consistency. As may be appreciated, without needing to coordinate with another person, the medical professional may perform a procedure with greater accuracy based on his or her own senses and perceptions and reactions thereto. Moreover, training of both a physician as well as a medical assistant is no longer necessary.

In some aspects, devices and systems of the present disclosure are configured for minimally-invasive surgical procedures, such as transluminal, transcatheter, endoscopic, etc., procedures through a body passage. It will be appreciated that reference to a body passage includes naturally-existing passages (e.g., the colon) as well as medically-created passages (e.g., a passage created with the use of a medical instrument, and not existing without medical intervention) or otherwise. As may be appreciated, such devices may range from approximately one (1) foot (30.5 cm) to approximately six (6) feet (almost 2 meters), such as determined by the type of procedure and the part of the patient being accessed transluminally. In some aspects, a deployment device formed in accordance with various principles of the present disclosure may have a shorter device length than that of prior devices, such as because the device need not be accessed by a medical assistant (who is usually positioned further from the patient than the physician performing the procedure). Such reduction in device length may allow torque and force to be transmitted more efficiently along the length of the device and to the medical device at the distal end (e.g., implantable medical device or otherwise).

The shorter length of devices formed in accordance with various principles of the present disclosure (compared to prior devices) may also reduce friction and other force transmission losses.

Additionally or alternatively, the profile of the user engagement portion of a deployment device formed in accordance with various principles of the present disclosure may be streamlined for operation by a single hand of a medical professional. For instance, the proximal end may have a narrower outer diameter than other devices which may be used for similar procedures. The reduction in outer diameter may allow the medial professional to deliver more distal turns per user action (e.g., achieve more rotations with fewer manual movements) than larger diameter handles of prior devices. In some aspects, the deployment device may provide more than one user engagement interface of different diameters to allow for choice of greater torque application with fewer rotations effected by a given manual motion (a larger diameter user engagement interface), or more rotations effected by the same manual motion but with less torque applied (a smaller diameter user engagement interface).

In one example of an embodiment formed in accordance with various principles of the present disclosure, a deployment device and/or system is configured to deliver an implantable medical device to an implant site, to further manipulate the implantable medical device to be secured and implanted with respect to the implant site, and then to deploy and separate the implantable medical device from the deployment device and system. In accordance with various principles of the present disclosure, control of all, or nearly all, operations of the deployment device and/or system (e.g., to achieve advancement, engagement, implantation, separation, etc., of the implantable medical device) may be performed by a single medical professional. For instance, the components or elements of the deployment device and/or system which effect various movements of the implantable medical device (e.g., axially advance, axially retract, rotate, rotationally advance, rotationally retract, engage, implant, separate, etc.) may be positioned to be graspable by a single user, such as by a single hand of a single user, so that the user’s other hand may operate another medical device for use during the procedure. In some aspects, the medical device is a medical delivery device, such as a tubular member, catheter, introducer sheath, medical scope, etc.

In some aspects, a deployment device and/or system includes one or more components operably coupled with an implantable medical device to manipulate the implantable medical device. Movements of the one or more components of the deployment device and/or system may be controlled by a single user, and/or within a single hand of a user, such as by grasping the one or more components in one hand and linearly and/or rotationally moving the one or more components to linearly and/or rotationally move (linearly and/or rotatably) an implantable medical device. In some aspects, a driver element of the deployment device moves an implantable medical device, such as to rotate the implantable medical device into engagement with (e.g., initial engagement) and secure engagement with (e.g., anchored or otherwise secured or affixed with respect to) an implant site (e.g., a treatment site at which the implantable medical device is to be secured and deployed). In some aspects, different movements of the driver are needed to perform different movements and actions of the implantable medical device. For instance, an implantable medical device in the form of a helical anchor or tack is rotated into secure engagement with an anatomical structure (e.g., tissue) by rotation of the driver. The driver is then moved axially with respect to the helical anchor or tack to release the helical anchor or tack from the driver to be left deployed with respect to the implant site. In some aspects, the same manual actuator element may be moved to effect all movements of the driver, including both the rotational as well as the axial movements. In some aspects, the manual actuator element is configured to be grasped within and operated by a single hand.

Various embodiments of deployment devices, systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

In the drawings, it will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments are generally designated with the same reference numbers increased by a multiple of 1000 and redundant description is generally omitted for the sake of brevity. Moreover, certain features in one embodiment may be used across different embodiments and are not necessarily individually labeled when appearing in different embodiments.

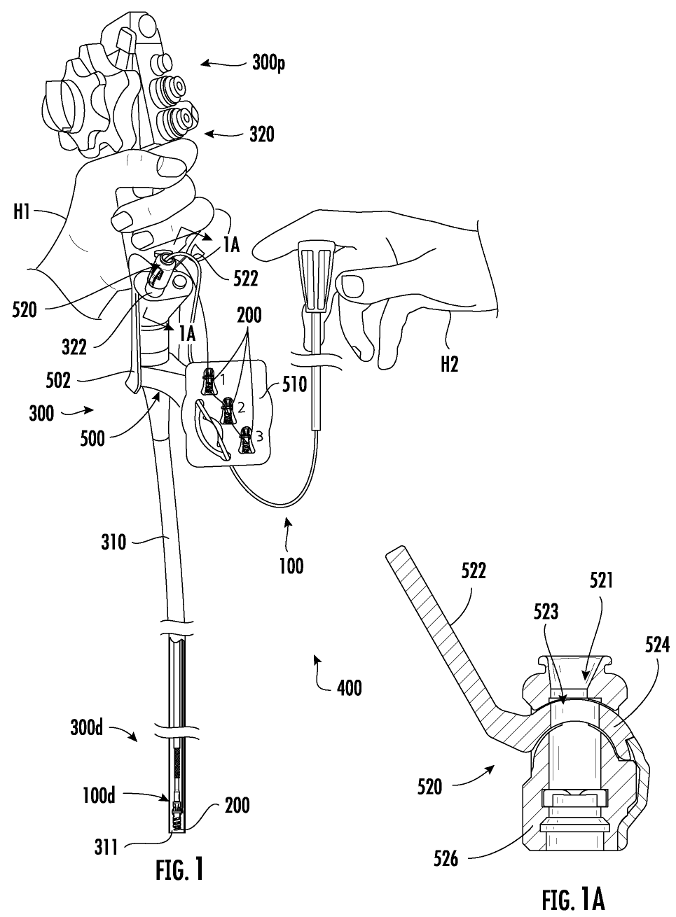

Turning now to the drawings, an example of an embodiment of a deployment device 100 formed in accordance with various principles of the present disclosure to deliver and deploy an implantable medical device 200 at an implant site is illustrated in FIGS. 1, 2, 3A, 3B, and 4.

Another medical device may be used during the procedure with which the deployment device 100 is used, such as in conjunction with the procedure. For instance, in some aspects, the other medical device is a separate and independent medical delivery device 300 with or by which the deployment device 100 may be delivered, such as illustrated in FIG. 1. In some aspects, the medical delivery device 300 is usable on its own and for procedures other than deployment of an implantable medical device 200. For instance, in some aspects, the medical delivery device 300 is configured to deliver one or more other medical devices (e.g., through one or more working channels defined through a flexible tubular elongate member 310 of the medical delivery device 300 which is insertable into the patient), irrigation fluid, vacuum pressure (suction), a visualization device (e.g., a camera, optic fiber, etc.), a light, etc. In some aspects, the medical delivery device 300 is a medical scope, such as an endoscope, gastroscope, colonoscope, duodenoscope, laparoscope, ureteroscope, arthroscope, cystoscope, bronchoscope, hysteroscope, etc. In some aspects, the medical delivery device 300 may include a flexible tubular elongate member 310 with a lumen 311 (e.g., working channel) defined therein configured to deliver the deployment device 100 therein and therethrough. In some aspects, the flexible tubular elongate member 310 is sized for insertion into a patient through a natural orifice or small incision and is sufficiently flexible to be navigated within curved or tortuous pathways within a patient’s body to deliver the distal end 100d of the deployment device 100 to an implant site within a patient’s body without the need for open surgery. As will be appreciated, at least a distal portion of the deployment device 100 is also sufficiently flexible to be navigated within curved or tortuous pathways within a patient’s body to deliver an implantable medical device 200 operably coupled to the distal end 100d of the deployment device 100 to an implant site within a patient’s body without the need for open surgery. In some aspects, the medical delivery device 300 includes a control handle 320, at a proximal end 300p of the medical delivery device 300, which may be used to control navigation of the flexible tubular elongate member 310 (at least the distal end 300d of the medical delivery device 300), and which may also control navigation of the distal end 100d of the deployment device 100 within the flexible tubular elongate member 310.

In some aspects, a deployment device 100 and medical delivery device 300 and optionally one or more additional medical instruments associated therewith may together be considered a deployment system 400. When delivered with a medical delivery device 300 such as described above, the distal end 100d of the deployment device 100 may be inserted into the working channel 311 of the flexible tubular elongate member 310 of the medical delivery device 300 via a port 322 defined in the control handle 320 and in operable communication with the working channel 311. In some aspects, one or more implantable medical devices 200 may be mounted on or otherwise operably coupled with the medical delivery device 300, such as with respect to the control handle 320 of the medical delivery device 300, such as to be conveniently accessible during a procedure. In the example of an embodiment illustrated in FIG. 1, a mounting bracket 500 may be used to mount or otherwise operably couple one or more implantable medical devices 200 with respect to the medical delivery device 300, such as with respect to the control handle 320 of the medical delivery device 300. Such operable coupling may allow for subsequent removability of the mounting bracket 500, such as after the procedure has been performed with the deployment device 100. The mounting bracket 500 may be any configuration known to those of ordinary skill in the art for securing a medical device to another medical device. For instance, the mounting bracket 500 may be configured to be engaged with the control handle 320 of the medical delivery device 300, such as by being snapped on or otherwise operably coupled or secured to the medical delivery device 300 (e.g., with respect to the control handle 320) to engage and be held in place with respect to the medical delivery device 300. In some aspects, further securement devices 502 may be used to secure the mounting bracket 500 with respect to the medical delivery device 300. For instance, the securement device 502 may be a strap or band or buckle which is wrapped around the medical delivery device 300, with at least one end secured to the mounting bracket 500 and/or the medical delivery device 300. In some aspects, the implantable medical devices 200 are themselves mounted on a device mount 510 which is then operably coupled with the mounting bracket 500. In the example of an embodiment illustrated in FIG. 1, the implantable medical devices 200 are relatively small, and are mounted on a device mount 510 in the form of a mounting card (e.g., a die card or otherwise relative stiff platform with respect to more than one implantable medical device 200 may be removably operably coupled).

When a deployment device 100 of the present disclosure is used in conjunction with (or at least generally at the same time as) another medical device, the user typically will hold one medical device in one hand and the other medical device in the other hand. For instance, in a minimally invasive procedure, a medical scope, such as the example of an embodiment of a medical delivery device 300 illustrated in FIG. 1, may be used to facilitate transluminal access to and viewing of a treatment site within the patient’s body. The medical professional performing the procedure typically holds the medical delivery device 300 in one hand H1 (typically the left hand) and operates one or more medical instruments with which the procedure is to be performed in the other hand H2 (typically the right hand). Typically, primary operation (e.g., navigation of the flexible tubular elongate member 310) of the medical delivery device 300 (e.g., medical scope) is performed by the medical professional performing the procedure. Often, medical instruments used in conjunction with the medical delivery device 300 have more than one component or element (such terms being usable interchangeably herein without intent to limit) which is moved to operate the medical instrument.

In accordance with various principles of the present disclosure, the proximal end 100p of a deployment device 100 is configured to be operated by a single medical professional and/or in a single hand H2 of the operator (e.g., medical professional, and typically the physician performing the procedure involving the deployment of the implantable medical device 200). Therefore, the medical professional may operate the medical delivery device 300 with a first hand H1, and may operate all elements of a deployment device 100 formed in accordance with various principles of the present disclosure (e.g., has access to full functionality of the deployment device 100) with his/her other hand H2. A deployment device 100 formed in accordance with various principles of the present disclosure may require elements thereof to be moved in more than one direction (e.g., axially along one or more axes, rotationally around one or more axes, etc.). In the example of an embodiment illustrated in FIG. 1, the deployment device 100 has a proximal end 100p configured to be grasped by a user’s hand H2 and maneuvered by one or more of the fingers of the user’s hand H2, and/or the palm of user’s hand H2, and/or the full hand H2. The movements of the elements of the deployment device 100 and the movements of the user’s hand H2 will generally depend on the configuration of the implantable medical device 200 and/or its connection with the deployment device 100 and/or other elements of the deployment device 100 or deployment system 400 in general. The proximal end 100p of the deployment device 100 of the present disclosure is configured so that elements of the deployment device 100 which are to be moved to operate the deployment device 100 are all accessible while grasped by the user’s hand H2. Optionally one or more elements or a portion of the elements of the deployment device 100 are readily grasped by the user’s other hand H1 while also grasping the medical delivery device 300 in that other hand H1 so that even if single-handed operation of the deployment device 100 is not performed, a single user may still fully operate the deployment device 100 without the need for assistance by another medical professional. Various operations may be performed by the deployment device 100 to deliver and deploy an implantable medical device 200 with respect to an implant site, such as moving the implantable medical device 200 to engage the implant site, securing the implantable medical device 200 with respect to the implant site, and deploying the implantable medical device 200 with respect to the implant site (including, without limitation, releasing the implantable medical device 200 from the deployment device 100 and/or leaving the implantable medical device 200 in a use position with respect to the implant site). One or more user engagement features are operably associated (one-to-one, or one to a plurality, or a plurality to one) with elements of the deployment device 100, and are configured to be manually engaged and operated (e.g., moved) by the user to effect the various operations of the deployment device 100. The various elements of the deployment device 100 which are operated by the medical professional may be referenced herein by various terms such as controllers, actuators, drivers, etc., without intent to limit. In accordance with various principles of the present disclosure, all such user engagement features are provided and positioned along the proximal end 100p of the deployment device 100 for engagement by or in a single hand of a single user or at least by a single user. A single medical professional thus need not rely on a medical assistant, and may perform the procedure without the need to communicate action requests to the medical assistant (to control an operation or function or aspect of the deployment device 100). Moreover, the user may perform the procedure without needing to put down the medical delivery device 300 (or other instrument) held in the first hand H1 or the deployment device 100 held in the other hand H2. Efficiency and speed of performance of the procedure is thereby increased, as well as accuracy because of the elimination of most if not all intercommunication between the medical professional primarily responsible for performing the medical procedure and the medical professional who is assisting.

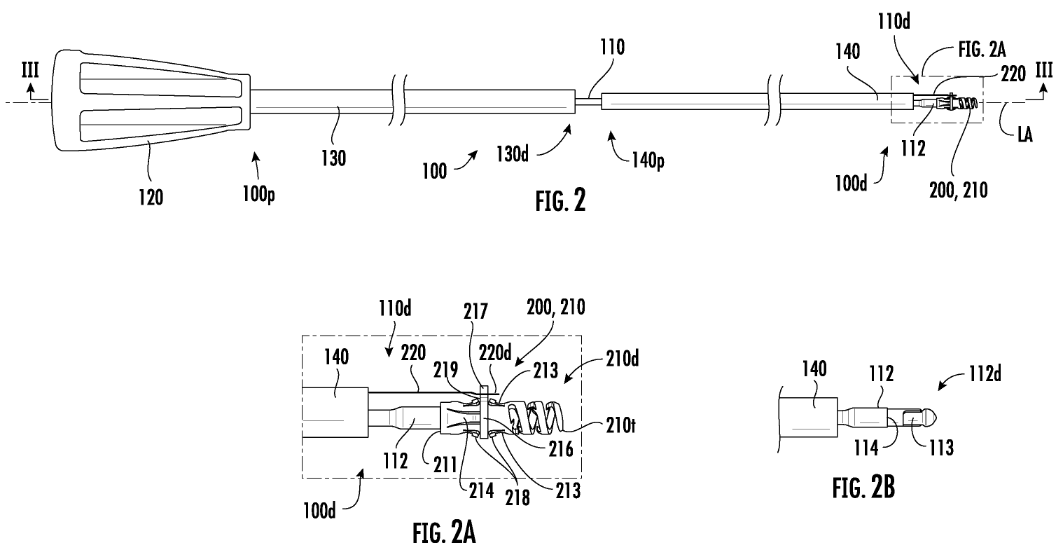

The configuration of a deployment device 100 to allow essentially one-handed and/or single-user operation in accordance with various principles of the present disclosure may be better appreciated with reference to a non-limiting example of an embodiment of an implantable medical device which may be deployed by the deployment device 100. Turning to FIG. 2, an example of an embodiment of an implantable medical device 200 which may be used with a deployment device 100 formed in accordance with various principles of the present disclosure is a helical anchor or tack 210 (referenced herein as a tack 210 for the sake of convenience and without intent to limit). However, it will be appreciated that principles of the present disclosure are not limited to applications or use with a helical tack 210, and a deployment device 100 formed in accordance with various principles of the present disclosure may be used to deliver, deploy, etc., other types, configurations, etc., of implantable medical devices 200.

As may be appreciated from the detail view in FIG. 2A of the distal end 100d of the example of an embodiment of a deployment device 100 illustrated in FIG. 2, the example of an embodiment of an implantable medical device 200 in the form of a tack 210 has a distal end 210d configured to engage an anatomical structure, such as tissue, to be implanted with respect to such anatomical structure. It will be appreciated that reference to engagement may encompass simple contact as well as secure engagement (e.g., including penetration into and/or anchoring with respect to the anatomical structure), and is not limited to one or the other unless specified. Moreover, reference to implanting includes secure engagement which allows the implantable medical device to be left in place, secured or otherwise anchored with respect to the implant site for the intended period of implantation, as may be determined by the device and/or the medical professional implanting the device. For the sake of convenience, and without intent to limit, reference is made herein to engagement and implantation with respect to an anatomical structure, and/or tissue, and/or an implant site interchangeably and without intent to limit. The distal end 210d of the tack 210 may have a tissue-penetrating tip 210t configured to penetrate tissue (e.g., sufficiently sharp to penetrate tissue) upon advancing the tack 210 distally towards the tissue with the use of the deployment device 100. In some aspects, the distal end 210d of the tack 210 presents a blunt surface upon axial contact with tissue, and the deployment device 100 simply axially advance into contact with the implant site, and further operation of the deployment device 100 is needed to cause the tack 210 to penetrate into tissue at the implant site. In some aspects, the tissue-penetrating tip 210t may be configured to pierce and/or penetrate tissue by rotation with respect to the tissue, such as by helical advancement, also with the use of the deployment device 100 of the present disclosure.

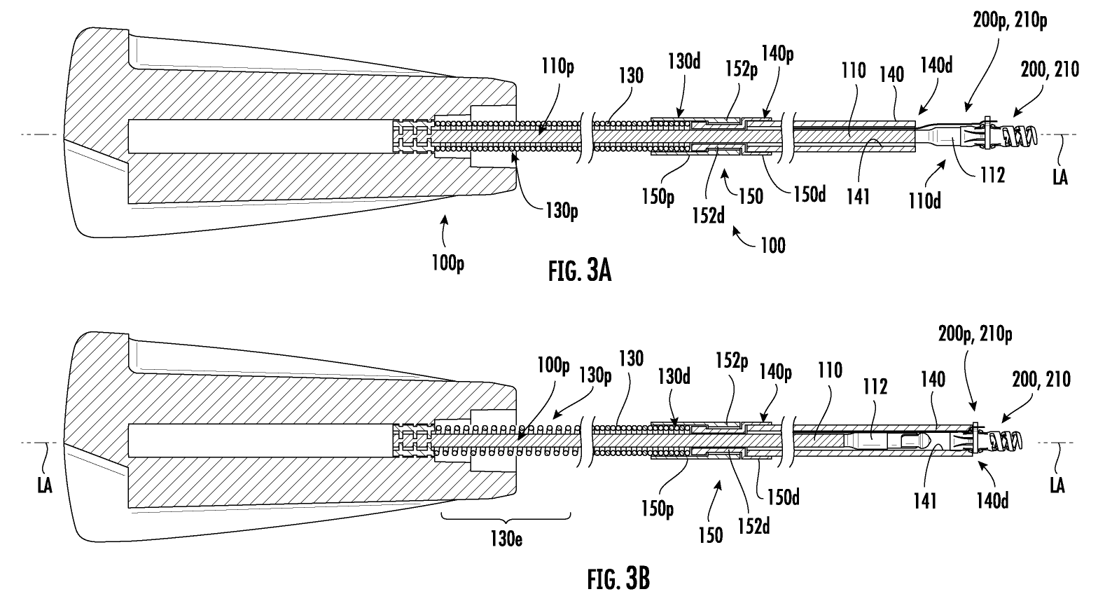

The example of an embodiment of a deployment device 100 illustrated in FIG. 2 includes a driver flexible elongate element 110 configured to be operably coupled with the tack 210. In some aspects, the driver flexible elongate element 110 is configured to be sufficiently flexible to be navigated through curved or tortuous pathways within a patient’s body to access an implant site, such as to deliver and/or deploy an implantable medical device 200 to an implant site. Moreover, if delivering an implantable medical device 200 in the form of a helical tack 210, the driver flexible elongate element 110 is not only axially advanceable and retractable within the patient’s body (and any delivery lumen through which the driver flexible elongate element 110 is advanced into the patient’s body), but also is rotatable within the patient’s body. Accordingly, in some aspects, the driver flexible elongate element 110 may be formed of a torqueable shaft, such as formed of a coiled wire, such as known to those of ordinary skill in the art. The proximal end 110p of the driver flexible elongate element 110 extends proximally toward the proximal end 100p of the deployment device 100 (as may be seen in the cross-sectional view in FIG. 3A, taken along line IIIA-IIIA of FIG. 2), for manual access by a user. The user may thus grasp the proximal end 110p of the driver flexible elongate element 110, directly or via another element with which the driver flexible elongate element 110 is operably coupled (as discussed in further detail below), to move the tack 210 axially along the longitudinal axis LA (of the tack 210 and/or the driver flexible elongate element 110), such as to axially advance or retract the tack 210, and/or to move the tack 210 rotationally about the longitudinal axis LA, such as to rotationally advance or retract the tack 210 with respect to the implant site. In the illustrated example of an embodiment, the proximal end 110p is operably coupled to a control handle 120 of the deployment device 100 for engagement by the user’s hand H2 (such as illustrated in FIG. 1).

In the example of an embodiment illustrated in FIG. 2, the distal end 110d of the driver flexible elongate element 110 includes a driver 112 which is configured to be operably coupled to the implantable medical device 200 to impart axial and/or rotational movement to the implantable medical device 200 to drive the implantable medical device 200 with respect to the implant site. It will be appreciated that the term drive is used herein broadly to include various actuation movements imparted to the implantable medical device 200 by the driver flexible elongate element 110 such as to move the implantable medical device 200. It will be appreciated that terms such as move, navigate, advance, retract, extend, engage, deploy, etc., including other grammatical forms thereof, may be usable interchangeably herein without intent to limit. In some aspects, the driver 112 and the implantable medical device 200 are configured to have corresponding features which allow the driver 112 to impart axial and/or rotational movement to the implantable medical device 200. For instance, various interengagement features (e.g., locking elements, stops, ribs, grooves, etc., such as known to those of ordinary skill in the art) may be provided to operably couple the driver 112 and the implantable medical device 200, such as to allow movement of the driver 112 to be imparted to the implantable medical device 200 (e.g., for movement of the driver 112 to cause a similar or different movement of the implantable medical device 200), as well as to allow optional separation of the implantable medical device 200 from the driver 112, such as to allow separation of the implantable medical device 200 from the deployment device 100 upon deployment of the implantable medical device 200.

In the example of an embodiment of an implantable medical device 200 illustrated in the form of a tack 210 in FIG. 2A, the tack 210 is formed from a tube (e.g., a laser cut tube) with a lumen 211 defined axially therethrough with a proximal opening for receiving the distal end 112d of the driver 112 (illustrated in FIG. 2B). The distal end 112d of the driver 112 may be formed with an increased diameter receivable in a corresponding recess 213 (e.g., apertures, a circumferential groove, etc.) defined in the wall of the tack 210 to inhibit inadvertent separation until deployment is complete and separation of the tack 210 from the deployment device 100 is desired. It will be appreciated that other configurations, such as a recess or circumferential groove on the exterior of the driver 112 and a corresponding radially-inwardly extending tab or rib within the lumen 211 of the tack 210, or other interengagement features known to those of ordinary skill in the art to inhibit or prevent relative axial movement between two components may be used, the present disclosure not being limited in this regard. In some aspects, a shoulder 114 may be provided to provide a stop to limit advancement of the driver 112 into the lumen 211 of the tack 210 (it will be appreciated that the relative proportions of the lumen 211 and the shoulder 114 may allow for such interference). Additionally, the driver 112 and tack 210 include interengagement features such as known by those of ordinary skill in the art to inhibit or prevent relative rotational movement therebetween so that rotation of the driver 112 causes rotation of the tack 210 to advance into tissue at the implant site. For instance, in the illustrated example of an embodiment, the driver 112 includes one or more axially-extending recesses 113 and the tack 210 includes one or more corresponding radially-inwardly-extending tabs 214 or extensions (e.g., formed by axially cutting the wall of the tack 210 and radially-inwardly deforming portions of the wall between a pair of cuts) which interengage to inhibit or prevent relative radial movement between the driver 112 and the tack 210. It will be appreciated that other configurations, such as a reverse arrangement (radially-outwardly extending axial ribs on the driver 112 and axially-extending grooves in the tack 210), a lock and key type interengagement, or other configuration known to those of ordinary skill in the art may be used to engage the driver 112 and the tack 210 in a non-rotatable manner, the present disclosure not being limited in this regard. A non-limiting example of an embodiment of a driver and tack which may be used with the deployment device 100 of the present disclosure is described in U.S. Patent 11,864,751 to Mitelberg, et al., issued Jan. 9, 2024, and titled Endoscopic Tissue Approximation System And Methods, the entire disclosure of which is hereby incorporated herein for all purposes.

In some aspects, an additional medical device may be secured with respect to the implant site by the tack 210. In the example of an embodiment illustrated in FIGS. 2 and 2A, a flexible elongate member 220, such as a suture or other element, is operably coupled with the tack 210 and may be secured via the tack 210 to the implant site at which the tack 210 is deployed. In some aspects, the flexible elongate member 220 is rotationally operably coupled with the tack 210 so that the tack 210 may be rotated independently of the flexible elongate member 220 to be advanced into and secured with respect to the implant site. In the example of an embodiment illustrated in FIG. 2A, the tack 210 includes a device coupler 216 which is rotatably mounted with respect to the wall of the tack 210 and configured to be operably coupled with the flexible elongate member 220 (e.g., a distal free end 220d of the flexible elongate member 220). In the example of an embodiment illustrated in FIG. 2A, the device coupler 216 includes an eyelet ring 217 which receives and retains the distal free end 220d of the flexible elongate member 220 and the device coupler 216 is rotatable with respect to the wall of the tack 210 so that rotation of the tack 100 does not cause tangling of the suture. In some aspects, the device coupler 216 is secured with respect to, engaged with respect to, mounted on, mounted circumferentially around or over, or otherwise retained with respect to wall of the tack 210 to be rotatable yet axially fixed with respect to the wall of the tack 210. In the example of an embodiment illustrated in FIG. 2A, the wall of the tack 210 defines radially-outwardly extending tabs 218 which define a channel 219 in which the device coupler 216 may be rotatably positioned and axially retained along the length of the tack 210.

As may be appreciated in view of the above, the driver 112 at the distal end 110d of the driver flexible elongate element 110 of the deployment device 100 is rotationally operably coupled with the tack 210 and manipulated by manual engagement (direct or indirect) with the proximal end 110p of the driver flexible elongate element 110. The implantable medical device 200 may thereby be advanced into engagement with and/or secured with respect to an implant site via manual manipulation of one of the user’s hands H2 while the other of the user’s hands H1 is holding and/or manipulating another device (such as the medical delivery device 300 illustrated in FIG. 1). In accordance with various principles of the present disclosure, the proximal end 100p of the deployment device 100 is configured to be manipulated by a single hand of a single user to operate the driver flexible elongate element 110 to move (advance, retract, rotate, etc.) an implantable medical device 200 such as the illustrated tack 210, to an implant site and into secure engagement with an implant site. For instance, the user may hold the control handle 120 in a palm of one hand and rotate the control handle 120 and/or a portion of the deployment device 100 adjacent but distal to the control handle 120 (e.g., reachable by the user’s fingers if the control handle 120 is in the palm of the user’s hand) to axially or rotationally operate the driver 112 to effect movement of the implantable medical device 200. As may be appreciated, the dimensions of the user engagement interface (control handle 120 or portion of the deployment device 100 distal to the control handle 120, such as the exterior of the actuator flexible elongate element 130) may affect the torque at and/or number of rotations of the tack 210 achieved by rotating the use engagement interface. As may be appreciated with reference to the cross-sectional views illustrated in FIGS. 3A and 3B, along line III-III of FIG. 2, the proximal end 110p of the driver flexible elongate element 110 may not be directly accessible, but, instead, may be manually accessed by the user via another element of the deployment device 100, such as an actuator flexible elongate element 130, as described in further detail below. Additionally or alternatively, the driver flexible elongate element 110 may not be directly coupled to the control handle 120 of the deployment device 100, but, instead, may be operably coupled to the control handle 120 via another element of the deployment device 100, such as the actuator flexible elongate element 130, as described in further detail below.

In the example of an embodiment of a deployment device 100 and implantable medical device 200 illustrated in and described above with reference to FIG. 2 and FIG. 2A, the tack 210 is deployed and separated from the driver 112 by relative movement between the driver 112 and the tack 210, typically opposite the direction in which the tack 210 was mounted on driver 112 and/or the driver 112 was received within the lumen 211 of the tack 210. Such movement is typically relative axial movement between the driver 112 and the tack 210. In some aspects, such as illustrated in FIGS. 2, 3A, 3A, 3B, and 4, the deployment device 100 includes a separator element 140 configured and/or positioned (e.g., mounted or positioned) adjacent the distal end 100d of the deployment device 100 and configured to interact with the tack 210 and/or the driver 112 to move the tack 210 and the driver 112 with respect to each other to cause the tack 210 to separate from the driver 112. In some aspects, the separator element 140 is the distal end of a flexible elongate tubular element (defining a lumen 141 through which the driver flexible elongate element 110 extends, as illustrated in FIGS. 3A and 3B), as described in further detail below, although the present disclosure need not be limited in this regard, and other configurations are within the scope of the present disclosure. In some aspects, the separator element 140 is configured and/or positioned to allow movement of the driver 112 with respect to the separator element 140 to push the tack 210 distally off the distal end 100d of the driver flexible elongate element 110.

In accordance with various principles of the present disclosure, the axial movement of the deployment device 100 to advance an implantable medical device 200 to an implant site, the rotational movement to rotationally drive an implantable medical device 200 in the form of a helical tack 210 into tissue at the implant site, and the axial movement to deploy the tack 210 and optionally separate the tack 210 from the deployment device 100 may all be manually controlled by a single hand of a single user. As noted above, the deployment device 100 illustrated in FIGS. 2, 3A, 3B, and 4, includes an actuator flexible elongate element 130 which is operably associated with the driver flexible elongate element 110. In accordance with various principles of the present disclosure, the actuator flexible elongate element 130 is fixedly operably coupled with the driver flexible elongate element 110 so that the actuator flexible elongate element 130 and the driver flexible elongate element 110 rotate and move axially together. In some aspects, the user manually grasps the proximal end 130p of the actuator flexible elongate element 130 to move (axially and/or rotationally) the actuator flexible elongate element 130 to cause similar movement of the driver flexible elongate element 110. In some aspects, the proximal end 130p of the actuator flexible elongate element 130 may be fixedly coupled with the control handle 120 of the deployment device 100 such that movement of the control handle 120 is imparted to the actuator flexible elongate element 130, and also to the driver flexible elongate element 110 (by virtue of the operable coupling of the driver flexible elongate element 110 and the actuator flexible elongate element 130). In some aspects, the proximal end 130p of the actuator flexible elongate element 130 is fixedly operably coupled with the proximal end 110p of the driver flexible elongate element 110 so that the actuator flexible elongate element 130 and the driver flexible elongate element 110 rotate and move axially together. It will be appreciated that reference to the proximal ends 110p and 130p need not be limited to the proximal-most ends, but simply an end that remains outside the patient’s body. In some aspects, the user may manually engage the actuator flexible elongate element 130 directly to move the actuator flexible elongate element 130 and thus the driver flexible elongate element 110 to control operation of the deployment device 100 to deliver and deploy an implantable medical device 200.

In order to cause the implantable medical device 200 to separate from the distal end 100d of the deployment device 100, as described above, the driver 112 and separator element 140 are moved with respect to each other, such as axially with respect to each other. In the example of an embodiment of a deployment device 100 illustrated in FIGS. 2, 3A, 3B, and 4, the separator element 140 is an elongate element which extends proximally from a distal end 140d which contacts the implantable medical device 200. In some aspects, the separator element 140 is sufficiently flexible to be navigated within curved or tortuous pathways within a patient’s body to reach an implant site at which the implantable medical device 200 is to be delivered. The relative dimensions (e.g., inner and outer diameters) of the separator element 140 and the implantable medical device 200 are selected such that relative movement of the separator element 140 and the driver 112 to move the distal end 140d of the separator element 140 closer to and then into contact with the proximal end 200p of the implantable medical device 200 causes the distal end 140d of the separator element 140 to push the implantable medical device 200 distally off the driver 112 upon further relative movement of the separator element 140 in the direction of the implantable medical device 200. As may be appreciated with reference to the example of an embodiment of an implantable medical device 200 illustrated in FIG. 2A, the dimensions of the distal end 140d of the separator element 140 and at least the proximal end 210p of the tack 210 are selected so that the distal end 140d of the separator element 140 can contact and push the tack 210 with sufficient force to overcome the above-described interengagement of the tack 210 and driver 112 to push the tack 210 distally off the driver 112. In some aspects, the separator element 140 is a tubular elongate element and the distal end 110d of the driver flexible elongate element 110 extends within a lumen 141 defined through the separator element 140. The separator element 140 may function as an outer bearing sleeve around the rotatable distal end 100d of the driver flexible elongate element 110 and/or the driver 112, such as to minimize whipping of the distal end 110d of the driver flexible elongate element 110 upon rotation thereof to rotate the driver 112.

In prior devices, an element such as the separator element illustrated in FIGS. 2, 2A, 2B, 3A, 3B, and 4, extends proximally to a proximal end of the deployment device so that the separator element and a driver may be moved independently and with respect to each other to effect separation of the implantable medical device from the driver. However, such configurations typically require use of more than one hand and/or readjustment of the position of the proximal end of the deployment device within the user’s hand (which also typically requires the use of more than one hand), and often the assistance of another medical professional to achieve separation of the implantable medical device from the deployment device.

In contrast, in accordance with various principles of the present disclosure, the actuator flexible elongate element 130 not only may be rotated to rotate the driver 112 to engage an implantable medical device 200 with tissue at an implant site, but may also be moved axially to axially move the driver 112 with respect to the separator element 140 to cause separation of the implantable medical device 200 from the driver 112. As noted above, the actuator flexible elongate element 130 may be manually grasped along a proximal end 100p of the deployment device 100 to cause rotation of the driver 112. Likewise, the actuator flexible elongate element 130 may be manually grasped along a proximal end 100pof the deployment device 100 to cause axial movement of the driver 112 with respect to the separator element 140. As such, a single user may manually control the deployment device 100 to effect all actions needed to deliver and deploy an implantable medical device 200 in a single hand or at least by a single user / operator / medical professional, leaving the other hand available to control another device, such as the medical delivery device 300 illustrated in FIG. 1. As may be appreciated, because the actuator flexible elongate element 130 is operably coupled with the driver flexible elongate element 110 to cause rotational movement of the driver flexible elongate element 110 with the same hand which controls axial movement of the driver flexible elongate element 110, the configuration as well as use of the deployment device 100 are streamlined and simplified (e.g., fewer components, only one operator, etc.).

It will be appreciated that various challenges may be presented in moving a driver 112 of the deployment device 100 with respect to a separator element 140 of the deployment device 100 because at least one of the driver 112 or the separator element 140 typically must be held against moving while the other moves, and/or both the driver 112 and the separator element 140 must be held separately (e.g., by different parts of a user’s hand or by different hands). Moreover, the distal end 140d of the separator element 140 must be positioned within the patient’s body adjacent to the implantable medical device 200 and the implant site, presenting further challenges to access thereto to hold the separator element 140 against relative movement with respect to the driver 112.

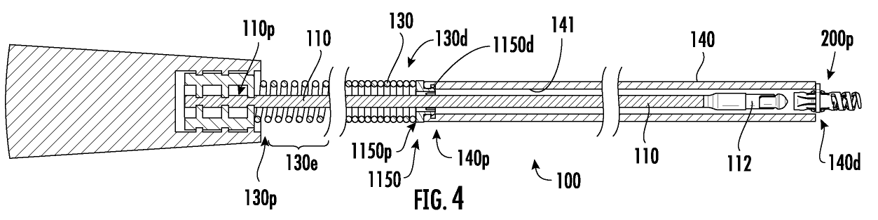

In accordance with various principles of the present disclosure, the actuator flexible elongate element 130 may be operably coupled with respect to the separator element 140 so that an exterior portion of the actuator flexible elongate element 130 which is accessible outside the patient’s body may be manually grasped and held to allow relative movement between the separator element 140 and the driver flexible elongate element 110. In some aspects, the separator element 140 is formed along a distal end 130d of the actuator flexible elongate element 130. In some aspects, the separator element 140 is operably coupled with respect to a distal end 130d of the actuator flexible elongate element 130. In some aspects, it may be desirable for the separator element 140 to be formed as a tubular bearing sleeve which “floats” over the driver flexible elongate element 110 to protect the lumen through which the driver flexible elongate element 110 extends from any whipping which may occur as a result of the rotational movement of the driver flexible elongate element 110 to rotationally advance or retract a helical tack 210 with respect to an implant site. In such case, the distal end 130d of the actuator flexible elongate element 130 may be operably coupled with the proximal end 140p of a separator element 140 in the form of a flexible elongate tubular element via a coupler 150, 1150 such as illustrated in FIGS. 3A, 3B, and 4. It is noted that although FIG. 3A is generally a cross-sectional view along line III-III of FIG. 2, the coupler 150 is optional and is not illustrated in FIG. 2 for the sake of simplicity as well as to illustrate an alternate arrangement of the elements of the deployment device 100. Moreover, it will be appreciated that although the outer diameter of the separator element 140 is illustrated as smaller than the outer diameter of the actuator flexible elongate element 130 in FIG. 2, the outer diameters of these elements may be substantially the same, as illustrated in FIGS. 3A, 3B, and 4.

The coupler 150 illustrated in FIGS. 3A and 3B and the coupler 1150 illustrated in FIG. 4 are each configured to operably couple the actuator flexible elongate element 130 and the separator element 140 so that axial movement of the actuator flexible elongate element 130 causes axial movement of the separator element 140. Thus, distal axial advancement of the actuator flexible elongate element 130 to advance the distal end 100d of the deployment device 100 distally into the patient to deliver the implantable medical device 200 to the implant site also distally axially advances the separator element 140. Similarly, proximal axial retraction of the actuator flexible elongate element 130 to retract the distal end 100d of the deployment device 100 proximally out of the patient to remove the deployment device 100 also proximally axially retracts the separator element 140. Moreover, in order to reduce torquing of the various elements of the implantable medical device 200, the coupler 150, 1150 may rotatably couple the actuator flexible elongate element 130 with the separator element 140 so that torquing of the actuator flexible elongate element 130 (to rotate the driver 112 via the driver flexible elongate element 110) is not also transmitted to the separator element 140. In some aspects, although the separator element 140 may provide a bearing sleeve for the distal end 110d of the driver flexible elongate element 110 (as noted above), an additional bearing sleeve may not be provided around the separator element 140. Relative rotation between the actuator flexible elongate element 130 and the separator element 140 may reduce unnecessary rotation of the separator element 140 (as the actuator flexible elongate element 130 rotates), and thus possible resultant effects of rotation of the separator element 140 (e.g., whipping). In some aspects, the coupler 150, 1150 may thus be considered a rotation joint.

In the example of an embodiment of a coupler 150 illustrated in FIGS. 3A and 3B, the coupler 150 includes a proximal coupler element 150p operably coupled (e.g., fixed against axial and/or rotational movement) with respect to the distal end 130d of the actuator flexible elongate element 130, and a distal coupler element 150d operably coupled (e.g., fixed against axial and/or rotational movement) with respect to the proximal end 140p of the separator element 140. In some aspects, the proximal coupler element 150pand the distal coupler element 150d are rotatably operably coupled with each other to allow relative rotation of the actuator flexible elongate element 130 and the separator element 140. For instance, the proximal coupler element 150p may include a radially inwardly extending engagement element 152p (e.g., a rib or a plurality of projections) and the distal coupler element 150d may include a circumferential groove 152d configured to receive the radially inwardly extending engagement element 152p of the proximal coupler element 150p. The reverse configuration, with a groove defined in the proximal coupler element 150p and a radially-outwardly extending engagement element on the distal coupler element 150d, may be implemented instead. Optionally, although not necessarily (for reasons as will become apparent), proximal coupler element 150p and the distal coupler element 150d are operably coupled with each other to inhibit or prevent relative axial movement of the actuator flexible elongate element 130 and the separator element 140.

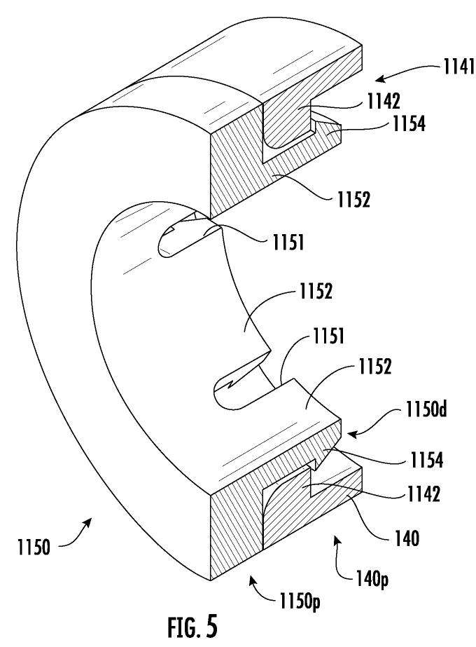

In the example of an embodiment of a deployment device 100 illustrated in FIG. 4, the coupler 1150 may be a single component with a proximal end 1150p fixed to the distal end 130d of the actuator flexible elongate element 130 and a distal end 1150d fitted into the lumen 141 of the separator element 140 (e.g., through which the driver flexible elongate element 110 may extend, as noted above) at the proximal end 140pof the separator element 140. As illustrated in further detail in FIG. 5, the coupler 1150 of FIG. 4 may have two or more proximally-extending flexible extensions or fingers 1152 which define radially-outwardly extending shoulders 1154 which may interengage with a radially-inwardly extending rib 142 defined along the lumen 141 of the separator element 140. The fingers 1152 may be separated by slits 1151 to allow the fingers 1152 to flex inwardly to be distally advanced past the rib 142 and then to return to their initial relaxed positions in which the fingers 1152 cannot be withdrawn proximally from their positions distal to the rib 142, such as to result in a snap fit of the fingers 1152 within the lumen 141 of the separator element 140. The interengagement of the fingers 1152 and the rib 142 may allow for relative rotational movement between the actuator flexible elongate element 130 and the separator element 140. For the sake of convenience, reference is hereinafter made to only the deployment device 100 illustrated in FIGS. 3A and 3B, it being appreciated that the descriptions are applicable to the deployment device 100 illustrated in FIG. 4 mutatis mutandis, and therefore such descriptions are not repeated for the sake of convenience brevity.

As noted above, at least a portion of the driver flexible elongate element 110 is fixed with respect to the actuator flexible elongate element 130 so that rotation of the actuator flexible elongate element 130 is imparted to the driver flexible elongate element 110 and the driver 112. Similarly, the operable coupling of the actuator flexible elongate element 130 and the driver flexible elongate element 110 allow axial movement of the actuator flexible elongate element 130 to be imparted to the driver flexible elongate element 110. Such operable coupling may be achieved in any of a variety of manners known to those of ordinary skill in the art, such as interengaging / interlocking mechanical elements (e.g., ribs and grooves, locks and keys, etc.), and/or chemical bonding (e.g., adhering with a material such as an adhesive, welding, overmolding, etc.), the present disclosure not being limited in this regard. As may be appreciated, in order to move the driver 112 axially with respect to the separator element 140, the actuator flexible elongate element 130 (which is fixedly coupled with respect to the driver flexible elongate element 110 and thus the driver 112) must be able to move axially with respect to the separator element 140. However, as described above, the actuator flexible elongate element 130 is also operably coupled with respect to the separator element 140 to impart axial movements thereto, such as to advance or retract the separator element 140 with advancement or retraction of the deployment device 100. In accordance with various principles of the present disclosure, at least a proximal portion of the actuator flexible elongate element 130 allows axial movement of the actuator flexible elongate element 130 relative to a distal portion of the actuator flexible elongate element 130 operably coupled with the separator element 140 (such as to be fixed against relative axial movement between the separator element 140 and the distal end 130d of the actuator flexible elongate element 130). For instance, a portion of the actuator flexible elongate element 130 accessible by the hand H2 which is used to operate the deployment device 100 (see, e.g., FIG. 1) is at least axially (and optionally also rotatably) movable with respect to a distal portion of the actuator flexible elongate element 130. In some aspects, a proximal end 130p of the actuator flexible elongate element 130 is axially (and optionally also rotatably movable with respect to a portion of the actuator flexible elongate element 130 distal to the proximal end 130p (e.g., spaced distally from the control handle 120 of the deployment device 100) and/or within the medical delivery device 300 and/or sufficiently adjacent the medical delivery device 300 to be accessible by the hand H1 which is used to operate the medical delivery device 300. It will be appreciated that the hand H2 and the hand H1 may be the hands of the same medical professional, thereby allowing manual control of both the deployment device 100 and the medical delivery device 300 (or other medical device) by the same medical professional.

In the example of an embodiment of an implantable medical device 200 illustrated in FIGS. 3A and 3B, the proximal portion of the actuator flexible elongate element 130 includes a separately axially movable portion 130e which allows a proximal portion of the actuator flexible elongate element 130 to be moved relative to a distal portion of the actuator flexible elongate element 130. In some aspects, the portion 130e is an elongatable portion 130e allowing a degree of elongation of at least a portion of the actuator flexible elongate element 130. It will be appreciated that terms such as elongatable, stretchable, expandable, extendable, extensible, etc., including other grammatical forms thereof, may be usable interchangeably herein without intent to limit. In some aspects, at least a portion of the actuator flexible elongate element 130 is formed from a coiled catheter (e.g., a wire coil catheter, such as formed of wire or an elastic plastic) which facilitates navigation through curved and/or tortuous passages, as well as torquing over extended lengths from a proximal end outside the body to a distal end inside the body and at a treatment site / implant site (over a length such as known to those of ordinary skill in the art). Such configuration allows a degree of axial extension (e.g., elongation) of a portion 130e of the actuator flexible elongate element 130 relative to the separator element 140 which is sufficient to cause axial movement of the driver flexible elongate element 110 and driver 112 relative to the separator element 140 to effect distal release of the implantable medical device 200 from the driver 112. The ability to elongate a portion 130e of the actuator flexible elongate element 130 allows axial movement of the proximal end 110p of the actuator flexible elongate element 130 even if the distal end 100d of the actuator flexible elongate element 130 is held against axial movement. In some aspects, the axially movable portion 130eis sufficiently elastically stretchable and resilient to return to its initial configuration (e.g., length) after deployment of an implantable medical device 200 so that the actuator flexible elongate element 130 may be returned to a known state and used again with the deployment device 100 to deploy another implantable medical device 200 (e.g., for loading of another implantable medical device 200 on the driver 112, advancing the implantable medical device 200 into the patient’s body, and deploying the implantable medical device 200 within the patient’s body). In some aspects, the extension of elongation may be tuned or adjusted (e.g., during manufacture or afterwards) to accommodate for the appropriate degree or amount of stretch to achieve the desired or necessary axial movement of the driver flexible elongate element 110 and driver 112, as well as a desired or necessary spring return (e.g., at least at the distal end 130d of the actuator flexible elongate element 130 and/or the distal end 110d of the driver flexible elongate element and/or the distal end 100d of the deployment device 100).

As noted above, the driver flexible elongate element 110 may be operably coupled with the actuator flexible elongate element 130 along a proximal portion of the actuator flexible elongate element 130. As such, axial movement of the actuator flexible elongate element 130 causes axial movement of the driver flexible elongate element 110 as well. A portion of the actuator flexible elongate element 130 distal to the relatively movable portion 130e of the actuator flexible elongate element 130 may be held in place axially (inhibited from axially moving) to hold the separator element 140 (which is operably coupled to the distal end 130d of the actuator flexible elongate element 130) against moving axially with respect to the driver flexible elongate element 110 (which is axially moved when the relatively movable portion 130e of the actuator flexible elongate element 130 is moved axially). As the distal portion of the actuator flexible elongate element 130 is held against axial movement, the proximal portion (e.g., the movable portion 130e) is moved (e.g., extended) proximally to retract the driver flexible elongate element 110 and the driver 112 proximally, and thereby to move the proximal end 210p of the tack 210 into engagement with the distal end 140d of the separator element 140 to cause the tack 210 to be distally pushed off and separated from the driver 112.

It will be appreciated that relative axial movement between the separator element 140 and distal end 130d of the actuator flexible elongate element 130 (coupled to the separator element 140) may be achieved in a variety of manners other than by elongating a portion 130e of the actuator flexible elongate element 130. For instance, a coupler 150, 1150 between the actuator flexible elongate element 130 and the separator element 140 may allow a degree of axial movement between the actuator flexible elongate element 130 and the separator element 140 to allow relative movement of the driver 112 (coupled to the actuator flexible elongate element 130 via the driver flexible elongate element 110) and the separator element 140 to effect separation of the driver 112 and the implantable medical device 200. It will be appreciated that various operable couplings other than those described herein may achieve the desired relative axial movement of the driver 112 and the separator element 140 in a manner which allows the actuator flexible elongate element 130 to be moved (axially and/or rotatably) in a single hand and/or by a single user in accordance with various principles of the present disclosure.

It will be appreciated that the distal portion of the actuator flexible elongate element 130 may be held in place axially in a variety of manners. For instance, a brake accessory 520 may be provided on the medical delivery device 300 for operation by the hand H1 of the user operating the medical delivery device 300. An example of an embodiment of a brake accessory 520 is illustrated in FIG. 1 as mounted on the port 322 defined in the control handle 320 to communicate with an access lumen of the port 322 (which, in turn, is in communication with the flexible tubular elongate member 310 of the medical delivery device 300 for delivery of at least a portion of the deployment device 100, along with the implantable medical device 200, therethrough). As illustrated in further detail in FIG. 1A, the brake accessory 520 defines a lumen 521 through which a portion of the actuator flexible elongate element 130 may be inserted (to extend into a lumen of the port 322 of the medical delivery device 300) to be further inserted into the patient’s body. The brake accessory 520 further includes an actuator 522 (such as in the form of a lever) movable to shift an opening 523 defined in a locking member 524 within a housing 526 of the brake accessory 520 in and out of alignment with the lumen 521 defined in the brake accessory 520. Movement of the opening 523 out of alignment with the lumen 521 of the brake accessory 520 holds the portion of the actuator flexible elongate element 130 extending through the lumen 521 and opening 523 against axial movement with respect to the housing 526. In some aspects, the actuator 522 of the brake accessory 520 may be readily actuated by a finger (e.g., a pinkie) of the hand H1 which holds the medical delivery device 300 so that a single user may hold the medical delivery device 300 in a first hand H1, with the deployment device 100 in the other hand H2, and still be able to cause the desired movement of the actuator flexible elongate element 130 relative to the separator element 140 to cause separation of the implantable medical device 200 from the driver 112. An example of a brake accessory 520 usable with the present disclosure is described in Provisional Patent application 63/790,850 [Attorney Docket 2001.3977100], titled INSTRUMENT ENGAGEMENT TOOL FOR AN ENDOSCOPE, and filed on Apr. 18, 2025, the entire disclosure of which is hereby incorporated herein for all purposes.

Additionally or alternatively, a distal portion of the actuator flexible elongate element 130 may be held axially in place manually by the hand H1 which operates the medical delivery device 300 (or other medical device), without the need for a brake accessory 520. For instance, the user’s hand H1 may be sufficiently close to the port 322 defined on the control handle 320 of the medical delivery device 300 to easily hold a portion of the actuator flexible elongate element 130 adjacent and outside the port 322. For instance, the control handle 520 may be held in the palm of the user’s hand H1 with a portion of the actuator flexible elongate element 130 (e.g., adjacent the port 522) between two fingers (e.g., the thumb and forefinger) of the hand H1. The user may thus restrict axial movement of a distal portion of the actuator flexible elongate element 130, and thus the separator element 140, with one hand H1 while axially moving a proximal portion of the actuator flexible elongate element 130 (e.g., the movable portion 130e). While retraining the portion of the actuator flexible elongate element 130 held / grasped by the hand H1 holding the medical delivery device 300, the user may axially retract the driver flexible elongate element 110 and driver 112 relative to the separator element 140 with the other hand H2 to cause separation of the implantable medical device 200 from the driver 112. A single user may thus actuate the deployment device 100 using one hand H2 to deploy and separate the implantable medical device 200 from the deployment device 100 while also controlling another medical device (e.g., a medical delivery device 300 as illustrated in FIG. 1) with the user’s other hand H1.

Additionally or alternatively, a portion of the actuator flexible elongate element 130 may be held axially in place manually by a portion of a hand (e.g., between two fingers, such as the thumb and forefinger) while the user moves a more proximal portion of the actuator flexible elongate element 130 (e.g., the movable portion 130e) proximally with another portion of the user’s hand (e.g., the palm and/or other fingers). Such action may be likened to using a single hand to remove a cap from a writing instrument. A single user may thus proximally retract the driver flexible elongate element 110 (by proximally axially moving a portion of the actuator flexible elongate element 130) while holding the separator element 140 from axial movement with respect to the driver flexible elongate element 110. A single user may thus actuate the deployment device 100 using the same hand to deploy and separate the implantable medical device 200 from the deployment device 100.

In some aspects, the coupler 150, 1150 may allow a degree of relative axial movement between the actuator flexible elongate element 130 and the separator element 140. In such configuration, the degree of relative movement may be sufficient to cause the separator element 140 to cause the implantable medical device 200 to be separated from the driver 112 without the need for a movable (e.g., expandable) portion 130e of the actuator flexible elongate element 130.

In some aspects, the deployment device 100 is streamlined by having a user engagement portion extending outside the body which is formed by the exterior of the actuator flexible elongate element 130, which itself is a relative thin element. For instance, in some aspects, the outer diameter of the actuator flexible elongate element 130 may be less than approximately 3.2 mm (e.g., if delivered through a medical scope with a working channel having an inner diameter of 3.2 mm, as is common in lower gastrointestinal medical scopes), or even less than approximately 2.8 mm (e.g., if delivered through a medical scope with a working channel having an inner diameter of 2.8 mm, as is common in upper gastrointestinal medical scopes). Even if a control handle 120 is provided as a manual user engagement feature, the control handle 120 need not have further moving parts, and may be generally streamlined with an ergonomic and/or uncomplicated shape, profile, configuration, etc. The user may manipulate only the exterior of the proximal end 130p of the actuator flexible elongate element 130 or the control handle 120 to control various movements of the deployment device 100 (e.g., of the driver 112) to effect delivery, deployment, and optional separation of the implantable medical device 200.

It will be appreciated that although principles of the present disclosure have been described with reference to deployment of an implantable medical device in the form of an anchor or tack, the present disclosure need not be limited in this regard. For instance, principles of the present disclosure may be applied to control of different devices, such as clips, graspers, scissors, etc. Principles of the present disclosure may be particularly advantageously applied to device which require movement in more than direction, particularly if operated in conjunction with another medical device.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

Various further benefits of the various aspects, features, components, and structures of a deployment device and system and associated methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and / or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader’s understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A deployment device for deploying an implantable medical device at an implant site within a patient’s body, said deployment device comprising:
a driver flexible elongate element with a distal end including a driver configured to be operably coupled with an implantable medical device to deliver the implantable medical device to the implant site;
a separator element adjacent said driver and movable with respect to said driver to act upon the implantable medical device to separate the implantable medical device from said driver for deployment at the implant site; and
an actuator flexible elongate element operably coupled with respect to said driver flexible elongate element to move axially and rotationally with said driver flexible elongate element, and axially movable with said separator element to advance said separator element to the implant site;
wherein at least a portion of said actuator flexible elongate element is axially movable with respect to said separator element to cause relative movement between said driver and said separator element to cause separation of the implantable medical device from said driver.

2. The deployment device of claim 1, wherein said actuator flexible elongate element is operably coupled with respect to said separator element to move said separator element axially, and additionally is axially movable with respect to said separator element.

3. The deployment device of claim 1, wherein said actuator flexible elongate element is elongatable to move axially with respect to said separator element.

4. The deployment device of claim 1, wherein said actuator flexible elongate element is stretchable to move axially with respect to said separator element.

5. The deployment device of claim 1, further comprising a control handle operably coupled with said actuator flexible elongate element.

6. The deployment device of claim 5, wherein said driver flexible elongate element is operably coupled with said actuator flexible elongate element to be operably coupled with said control handle such that movement of said control handle moves said driver flexible elongate element.

7. The deployment device of claim 6, wherein a proximal portion of said actuator flexible elongate element adjacent and distal to said control handle is manually accessible for manual movement to cause movement of said driver flexible elongate element.

8. The deployment device of claim 7, wherein said control handle and the proximal portion of said actuator flexible elongate element are accessible in a single hand of a user.

9. The deployment device of claim 1, wherein said actuator flexible elongate element is rotatably coupled with respect to said separator element to rotate with respect to said separator element.

10. The deployment device of claim 9, wherein said actuator flexible elongate element is rotatably coupled with respect to said separator element via a coupler, and said at least a portion of said actuator flexible elongate element is axially movable with respect to said coupler.

11. A deployment system for deploying an implantable medical device at an implant site within a patient’s body, said deployment system comprising:
an actuator flexible elongate element having a proximal end and a distal end;
a driver operably coupled with respect to the distal end of said actuator flexible elongate element to move axially and/or rotationally with said actuator flexible elongate element; and
a separator element adjacent said driver;
wherein:
the proximal end of said actuator flexible elongate element is manually manipulable in a single hand of a user in more than one direction to advance and deploy an implantable medical device from said driver, as well as to cause the implantable medical device to separate from said driver to be left at the implantable medical device implanted with respect to the implant site within the patient’s body.

12. The deployment system of claim 11, further comprising a control handle operably coupled with the proximal end of said actuator flexible elongate element and sized to fit within a user’s hand and allow the user’s fingers to contact a proximal portion of said actuator flexible elongate element adjacent and distal to said control handle.

13. The deployment system of claim 12, wherein said control handle and said proximal portion of said actuator flexible elongate element adjacent and distal to said control handle are accessible in a single hand of a user.

14. The deployment system of claim 12, further comprising a brake accessory operably coupled with said actuator flexible elongate element to fix a portion of said actuator flexible elongate element distal to said control handle against axial movement with respect to said separator element.

15. The deployment system of claim 14, further comprising a medical delivery device having a port through which said driver, said separator element, and the distal end of said actuator flexible elongate element are deliverable into a patient’s body, wherein said brake accessory is mounted with respect to the port for operation by a user’s first hand operating said medical delivery device while the proximal end of said actuator flexible elongate element is graspable in the user’s second hand for operation of said actuator flexible elongate element.

16. A method of deploying an implantable medical device at an implant site within a patient’s body, said method comprising:
distally advancing the implantable medical device, operably coupled to a distal end of a driver of a deployment device, into a patient;
engaging the implantable medical device with the implant site;
deploying the implantable medical device with respect to the implant site; and
releasing the implantable medical device from the driver;
wherein advancing, engaging, deploying, and releasing are all performed by a single medical professional.

17. The method of claim 16, wherein advancing, engaging, deploying, and releasing are all performed by a single hand of a single medical professional.

18. The method of claim 17, further comprising:
advancing the implantable medical device through a medical delivery device and into the patient;
controlling the medical delivery device with a first hand of a single medical professional; and
controlling the driver of the deployment device with a second hand of the single medical professional.

19. The method of claim 16, further comprising axially advancing an actuator flexible elongate element with respect to a separator element positioned adjacent the driver, and axially moving the actuator flexible elongate element with respect to the separator element using the same hand.

20. The method of claim 19, further comprising advancing the implantable medical device into the patient’s body through a port of a medical delivery device, and holding a portion of an actuator flexible elongate element with the hand which holds the medical delivery device and a portion of the actuator flexible elongate element with the other hand, of the same medical professional, at least to release the implantable medical device from the driver.
